(19) United States
(12) Patent Application Publication
(10) Pub. No.: US 2018/0161020 A1
(43) Pub. Date: Jun. 14, 2018

(54) CLINICAL SAMPLING DEVICE

(71) Applicant: Caldbeck Overseas Limited, Tortola (VG)

(72) Inventor: Uri Friedlander, London (GB)

(73) Assignee: Caldbeck Overseas Limited, Tortola (VG)

(21) Appl. No.: 15/571,081

(22) PCT Filed: May 6, 2017
(86) PCT No.: PCT/IB2016/052600

§ 371 Date: Nov. 1, 2017

(30) Foreign Application Priority Data
May 6, 2015 (GB) 1507754.8

(51) Int. Cl. A61B 10/02

(52) U.S. Cl. CPC A61B 10/02

(57) ABSTRACT

A device is provided for the collection of a clinical sample from the body cavity of a patient. The device comprises a deformable sampling section. In use, the deformable sampling section expands radially to contact the walls of the cavity and collect the clinical sample.

### FIELD OF THE INVENTION

The present invention relates to a clinical sampling device for collecting clinical samples from a body cavity of a patient, as well as a deformable sampling section for use with a clinical sampling device, a kit providing such sampling sections and a method of preparing a clinical sample for analysis.

### BACKGROUND OF THE INVENTION

Colorectal cancer is a highly prevalent and often deadly oncological disease. It is the second leading cause of death from cancer in both the UK and in Europe. However, as the disease will often develop without identifiable symptoms, it is often only diagnosed at a late stage of the disease. Thus, detection of colorectal cancer at an early stage would greatly increase the chance of successful intervention and treatment.

At present, screening is most commonly achieved through faecal occult blood tests (FOBt) which, although cheap and simple, has a high rate of false positive and false negative results. However, alternative methods of diagnosis have been proposed based on exfoliated epithelial cells collected from the rectum.

Thus, there is a need for a cheap and simple to use device by which samples of epithelial cells can be collected the walls of the rectum, which is both inexpensive to manufacture and easy to use. There is also a need to provide a means by which a sample can be stored, without contamination, so as to ensure the integrity of screening results.

### SUMMARY OF THE INVENTION

The invention provides devices for collecting a clinical sample. Clinical samples may comprise tissues, cells or cellular material; micro-organisms such as bacteria, fungi and/or viruses; or biomarkers comprising nucleic acids and/or polypeptides, for example.

In one aspect of the invention, a device for collecting a clinical sample by contacting a sample site is provided, the device being movable between an undeployed configuration and a deployed configuration. The device comprises: a deformable sampling section extending along a longitudinal axis, wherein in the undeployed configuration the deformable sampling section has a first length in the longitudinal direction and a first width in a radial direction, perpendicular to the longitudinal axis, and in the deployed configuration, the deformable sampling section has a second length, shorter than the first length and at least part of the deformable sampling section has second width, greater than the first width, so as to contact the sample site.

That is to say, the overall length of the sampling section in the underployed configuration is greater than the overall length of the sampling section in the deployed configuration; and the maximum width of the sampling section in the undeployed configuration is less than the maximum width of the sampling section in the deployed configuration.

In other words, when transitioning from the undeployed configuration to the deployed configuration, the deformable sampling section is compressed so that at least part of the deformable sampling section expands radially.

In a further aspect of the invention, a device for collecting a clinical sample by contacting a sample site is provided, the device being movable between an undeployed configuration and a deployed configuration. The device comprises a deformable sampling section extending along a longitudinal axis and comprising at least one sampling edge, wherein in the undeployed configuration the deformable sampling section has a first width in a radial direction, perpendicular to the longitudinal axis, and the at least one sampling edge is not exposed and cannot contact the sample site; and in the deployed configuration, at least part of the deformable sampling section has a second width, greater than the first width, such that the at least one sampling edge is exposed and can contact the sample site.

That is to say, the maximum width of the sampling section in the undeployed configuration is less than the maximum width of the sampling section in the deployed configuration.

In other words, when transitioning from the undeployed configuration to the deployed configuration, at least part of the deformable sampling section expands radially such that the at least one sampling edge becomes exposed.

The first width of the sampling section, when the device is in its undeployed configuration, may be, for example, between 5 mm and 50 mm, or may be between 10 mm and 25 mm, for example. The first width of the sampling section, when the device is in its undeployed configuration may be, for example, 16 mm.

The second width of the sampling section, when the device is in its deployed configuration, may be, for example, between 10 mm and 100 mm, or may be between 20 mm and 50 mm, for example. The second width of the sampling section, when the device is in its deployed configuration may be, for example, 32 mm.

The first and second widths of the sampling section may be any combination of the first and second widths disclosed herein, provided that the second width is greater than the first width. For example, the first width may be between 5 mm and 50 mm and the second width may be between 10 mm and 100 mm, or between 20 mm and 50 mm. In another embodiment, the first width may be between 10 mm and 25 mm and the second width may be between 10 mm and 100 mm, or between 20 mm and 50 mm. Preferably, the first width may be 16 mm and the second width may be 32 mm.

The first length of the sampling section, when the device is in its undeployed configuration, may be, for example, between 10 mm and 100 m, or may be between 30 mm and 80 mm, for example. The first length of the sampling section, when the device is in its undeployed configuration may be, for example, 60 mm.

The second length of the sampling section, when the device is in its deployed configuration may be, for example, between 5 mm and 90 mm, or may be between 20 mm and 60 mm, for example. The second length of the sampling section, when the device is in its deployed configuration may be, for example, 40 mm.

The first and second lengths of the sampling section may be any combination of the first and second lengths disclosed herein, provided that the second length is shorter than the first length. For example, the first length may be between 10 mm and 100 mm and the second length may be between 5 mm and 90 mm. In another embodiment, for example, the first length may be between 30 mm and 80 mm and the second length may be between 20 mm and 60 mm. Preferably, the first length may be 60 mm and the second length may be 40 mm.

Because of the deformable nature of the sampling section, the device may conveniently and easily be introduced into a body cavity in the undeployed configuration, before being deployed internally into the deployed configuration to obtain a clinical sample by exfoliating the surface of the body cavity.

In some embodiments, the sampling section may comprise at least one fin. Fins are provided on the device to increase the exfoliation of cells from the surface of the body cavity when obtaining a clinical sample.

In some embodiments, the sampling section may comprise a plurality of fins, which are attached at their distal ends, are attached at their proximal ends, and which are separable therebetween.

The sampling section may comprise, for example, between 1 and 20 fins, 4 to 15 fins or may comprise between 5 and 10 fins, for example. The sampling section may comprise, for example, 6 fins.

The plurality of fins may be uniform, or they may not be uniform. That is to say, each fin of the plurality of fins may be substantially the same as the other fins of the plurality of fins, or the fins may differ in at least one respect. For example, the fins may be of different sizes, be of different thicknesses, have different degrees of deformability or may differ in some other respect.

In some embodiments, at least one fin or at least one of the plurality of fins may comprise a sampling edge which, when the device is in its deployed configuration, is exposed and, when the device is in its undeployed configuration, is not exposed. Thus, when the device is deployed in use to obtain a clinical sample, the sample may be collected on the exposed sampling edge. When the device is returned to the undeployed configuration, the clinical sample is contained within the closed sampling section thereby protecting the sample from contamination and loss of sample material as the device is removed from the body cavity.

In some embodiments, the at least one fin or at least one of the plurality of fins may comprise the sampling edge.

In some embodiments, the sampling edge may be disposed on an edge of the fin, extending longitudinally.

In some embodiments, when the device is in its undeployed configuration, the sampling edge may abut a second sampling edge. The second sampling edge may be disposed on a second fin of the plurality of fins.

In some embodiments, the at least one fin or at least one of the plurality of fins may comprise at least one rib. In one embodiment, at least one fin or at least one of the plurality of fins may comprise 1 to 10 ribs, 2 to 8 ribs or 3 to 5 ribs. Each fin in the plurality of fins may comprise the same or a different number of ribs, or may be devoid of ribs.

In some embodiments, the at least one rib may extend longitudinally along the fin. That is to say, the rib may extend in a longitudinal direction along at least part of the fin. The rib may be provided so that, in use, the rib may aid exfoliation of the sample site when the sampling section contacts the sample site.

In some embodiments, in the undeployed configuration, the sampling section may have a generally cylindrical configuration.

In any embodiment, the device may further comprise a puller member which at least partially extends through the sampling section along the longitudinal axis.

The puller member may take the form of a rigid or semi-rigid rod, for example. The puller member may take the form of a flexible wire or filament, for example.

In some embodiments, the puller member may be longitudinally inextendible.

In some embodiments, the puller member may cooperate with the distal end of the sampling section, so as to act upon the sampling section and longitudinally compress the sampling section when the puller member is moved in a proximal direction relative to the proximal end of the sampling section.

That is to say, the puller member may be such that it does not substantially extend in, for example, its longitudinal direction when a force acts upon it in that direction, and instead the puller member, being inextendible, will act upon the sampling section, the sampling section being deformable, so as to compress the sampling section longitudinally. Alternatively, the extendibility of the puller member and compressibility of the sampling section may be such that, for example, a longitudinal force acting upon the puller member will not result in substantial extension of the puller member but will instead result in compression of the sampling section, the sampling section being deformable and the puller member acting upon the distal end of the sampling section.

In some embodiments, the sampling section may be detachable from the puller member.

That is to say, the sampling section and puller member may be configured such that one of the sampling section and the puller member is removable from the other.

In some embodiments, the device may further comprise a sheath, proximal to the sampling section.

The sheath may be in the form of an elongate hollow body which extends along the longitudinal axis of the device. The sheath may be in the form of a hollow cylinder. The sheath may be dimensioned such that it has the same width as the deformable sampling section when the device is in its undeployed configuration.

In some embodiments, the puller member may also extend at least partially through the sheath.

In some embodiments, the sheath may have longitudinal compressibility less than that of the sampling section.

That is to say, the compressibility of the sheath and the compressibility of the sampling section may be such that, for example, a longitudinal force acting upon the sheath and deformable sampling section will not result in substantial compression of the sheath in the longitudinally direction but will result in compression of the sampling section in the longitudinal direction, the sheath having a compressibility less than that of the sampling section.

In some embodiments, the sheath may be longitudinally incompressible.

That is to say, the compressibility of the sheath and the compressibility of the sampling section may be such that, for example, a longitudinal force acting upon the sheath and deformable sampling section will not result in compression of the but will instead result in compression of the sampling section, the sheath having a compressibility less than that of the sampling section. In specific embodiments, the sheath may have a compressibility that is 0-5% of the compressibility of the sampling section, for example 0.5%-2% of the compressibility of the sampling section.

In some embodiments, the sheath and at least part of the sampling section may have different thicknesses.

That is to say, the sheath and at least part of the sampling section may, for example, be formed of the same, or different materials, those materials having a particular thickness. The thickness of the material which forms the sheath may be the same, similar, or dissimilar to the thickness of the material which forms the sampling section.

In some embodiments, the sheath and/or sampling section may be formed of two or more layers of different materials. For example, the sheath may be formed of two or more layers of different materials.

In certain embodiments, the thickness of the material which forms the sheath may be between 0.5 mm and 5 mm, for example 1 mm to 4 mm or 2 mm to 3 mm. The material forming at least part of the sampling section may have a thickness of between 0.1 mm and 3 mm, for example 0.5 mm to 2.5 mm.

The thickness of the sheath and at least part of the sampling section may be any combination of thicknesses disclosed herein, provided that at least part of the sampling section has a thickness less than that of the sheath. For example, the sheath may have a thickness of between 0.5 mm and 5 mm and the thickness of at least part of the sampling section may be between 0.1 mm and 2 mm. Preferably the sheath may have a thickness of 3 mm and at least part of the sampling section may have a thickness of 2 mm.

In some embodiments, the sheath may be integral with the sampling section.

That is to say, the sheath may be integral with the sampling section in that the sheath and sampling section are formed as one continuous piece. Alternatively, the sheath may be integral with the sampling section by way of the sheath and sampling section being attached to each other by way of bonding, welding or the like, so as to form a single piece.

In other embodiments, the sampling section may be separate from the sheath.

That is to say, the sampling section and sheath may be formed such that they are provided as separate, independent pieces.

In some embodiments, the sampling section may be detachable from the sheath.

That is to say, whether the sheath and sampling section are integral with, or separate from, each other, they may be configured so as to be separable, in other words, they may be configured such that one is removable from the other.

In some embodiments, the proximal end of the sheath may comprise a flange.

In some embodiments, the sheath may comprise at least one slot with a first portion which extends longitudinally along the sheath and the device further may comprise at least one protrusion which extends radially outwards from the puller member, through the slot, and is connected to the puller member.

The protrusions may, for example, be integrally formed with the puller member, or may be formed separately at attached or bonded to the rest of the puller member, or may be formed separately but interlocking with, or operably connected to, the puller member.

In some embodiments, the slot further comprises a second portion extending circumferentially from the first portion.

The slot and protrusion may, for example, be configured as a bayonet fitting.

In some embodiments, the slot and protrusion may be arranged to be able to retain the device in its deployed configuration.

That is to say, the slot and protrusion may be arranged such that the device can be moved into its deployed configuration, and the protrusion may then be moved relative to the slot such that the slot and protrusion will act to prevent the device from returning to its undeployed configuration. For example, the slot may be configured such that the puller member might be rotated, moving the protrusion circumferentially through the circumferential portion of the slot, so that the protrusion is prevented from moving in a distal direction, thereby preventing the puller member from moving in a distal direction, such that the device is prevented from returning to its undeployed configuration.

In some embodiments, the slot and protrusion may be arranged to be able to retain the device in its undeployed configuration.

That is to say, the slot and protrusion may be arranged such that the device can be moved into its undeployed configuration, and the protrusion may then be moved relative to the slot such that the slot and protrusion will act to prevent the device from returning to its deployed configuration. For example, the slot may be configured such that the puller member might be rotated, moving the protrusion circumferentially through the circumferential portion of the slot, so that the protrusion is prevented from moving in a proximal direction, thereby preventing the puller member from moving in a proximal direction, such that the device is prevented from returning to its deployed configuration.

In any embodiment, the deformable sampling section may be configured to bias the device to its undeployed configuration.

That is to say, the sampling section may be formed of a material which is deformable but which is, for example, also resilient so as to bias the device to its undeployed configuration.

In any embodiment, the deformable sampling section may be configured to bias the device to its deployed configuration.

That is to say, the sampling section may be formed of a material which is deformable but which is, for example, also resilient so as to bias the device to its deployed configuration.

In any embodiment, the sheath may be flexible.

In any embodiment, the puller member may be flexible.

In any embodiment, the device may be configured as a flexible catheter.

That is to say, the device may be configured with an extended, flexible body. For example, the sheath may form the extended flexible body.

In some embodiments, the device may comprise a plurality of sampling sections.

For example, the device may comprise anywhere between 2 and 5 sampling sections. The device may comprise 2, 3, 4 or 5 sampling sections. Preferably, the device may comprise 2 sampling sections.

In any embodiment, the sampling section may comprise silicone, ethylene propylene diene terpolymer (EPDM), thermoplastic elastomer (TPE) or natural latex rubber.

In any embodiment, the sampling section may be formed of any suitable material having a Young's modulus in the range 0.05-0.5 GPa.

In some embodiments, the sheath may comprise rigid or semi-rigid polypropylene (PP), polystyrene (PS) or polyethylene (PE).

In some embodiments, the sheath may be formed of any suitable material having a Young's modulus in the range 0.1-3.5 GPa.

In any embodiment, the puller member may comprise semi flexible PE or rigid PP.

In any embodiment, the sheath may be formed of any suitable material having a Young's modulus in the range 0.1-3.5 GPa.

Young's modulus is a type of elastic modulus and is a measure of the uni-axial strain in response to a uni-axial stress, within the range of stress for which the material behaves elastically. A preferred method of measuring the Young's modulus E is by means of measuring the transverse and longitudinal components of the speed of sound through the material, according to the equation E=ρ·C_{T}²(1+ν)(1−2ν)/(1−ν), where ν=(1−2(C_{T}/C_{L})²)/(2−2(C_{T}/C_{L})²), C_{L} and C_{T} are respectively the measured longitudinal and transverse speeds of sound through it and ρ is the density of the material. The longitudinal and transverse speeds of sound may be measured using ultrasonic waves, as is well known in the art.

In another aspect of the invention, a deformable sampling section for use in a clinical sample collecting device is provided, wherein, in use, the deformable sampling section has an undeployed configuration and a deployed configuration; wherein in the undeployed configuration the deformable sampling section has a first length in the longitudinal direction and a first width in a radial direction, perpendicular to the longitudinal axis, and in the deployed configuration, the deformable sampling section has a second length, shorter than the first length and at least part of the deformable sampling section has second width, greater than the first width, so as to contact a sample site.

In another aspect of the invention, a deformable sampling section is provided for use in a clinical sample collecting device, wherein, in use, the deformable sampling section has an undeployed configuration and a deployed configuration; wherein the deformable sampling section extends along a longitudinal axis and comprises at least one sampling edge; and wherein in the undeployed configuration the deformable sampling section has a first width in a radial direction, perpendicular to the longitudinal axis, and the at least one sampling edge is not exposed and cannot contact tissue; and in the deployed configuration, at least part of the deformable sampling section has a second width, greater than the first width, such that the at least one sampling edge is exposed and can contact a sample site.

In another aspect of the invention, a clinical sampling device is provided which is adapted to connect to a deformable sampling section, the device comprising: a sheath; and a puller member which at least partially extends through the sheath and is adapted to extend at least partially through the deformable sampling section, and the sheath and puller member are further adapted to act upon the deformable sampling section and longitudinally compress the sampling section when the puller member is moved in a proximal direction.

In another aspect of the invention, a kit is provided which comprises a deformable sampling section and a collection vessel, and optionally further comprises a device adapted to connect to the deformable sampling section.

In another aspect of the invention, a method of preparing a tissue sample for analysis is provided, wherein the tissue sample is disposed on a sampling section as set out above of a tissue sampling device as set out above, the method comprising:

inserting the device into a collection vessel,

detaching the sampling section from the puller member and/or sheath.

In some embodiments of the method, the step of detaching the sampling section from the puller member and/or sheath comprises expanding the at least part of the deformable sampling section to engage with the collection vessel.

In some embodiments, the method further comprises, before the step of inserting the device into the collection vessel, the step of introducing a transport buffer into the collection vessel.

In some embodiments, the method further comprises, after the step of detaching the sampling section from the puller member and/or sheath, the step of introducing a transport buffer into the collection vessel.

### BRIEF DESCRIPTION OF THE DRAWINGS

By way of example, embodiments of a tissue sampling device according to the invention will now be described with reference to the accompanying drawings:

FIG. 1 shows an exploded view of an embodiment of a clinical sampling device.

FIGS. 2a and 2b show the assembled clinical sampling device of FIG. 1 in an undeployed and deployed configuration, respectively.

FIG. 3 shows an exploded view of a further embodiment of a clinical sampling device.

FIGS. 4a and 4b show the assembled clinical sampling device of FIG. 3 in an undeployed and deployed configuration, respectively. FIGS. 4c and 4d show cross-section views of the device of FIG. 3 in an undeployed and deployed configuration, respectively.

FIGS. 5a and 5b show embodiments of a sampling section of a clinical sampling device.

FIG. 6 shows a clinical sampling device and a collection vessel.

FIG. 7 shows a cross-sectional view of a clinical sampling device and a collection vessel

FIG. 8 shows a further embodiment of a clinical sampling device.

FIGS. 9a and 9b show a further embodiment of a clinical sampling device in an undeployed and deployed configuration, respectively.

FIGS. 10a and 10b show cross-sectional views of the sampling section of the clinical sampling device of FIGS. 9a and 9b.

FIGS. 11a and 11b show a further embodiment of a clinical sampling device in an undeployed and deployed configuration, respectively.

FIG. 12a shows a further embodiment of a clinical sampling device. FIGS. 12b and 12c show cross-sectional and perspective views of the clinical sampling device shown in FIG. 12a, respectively.

### DETAILED DESCRIPTION

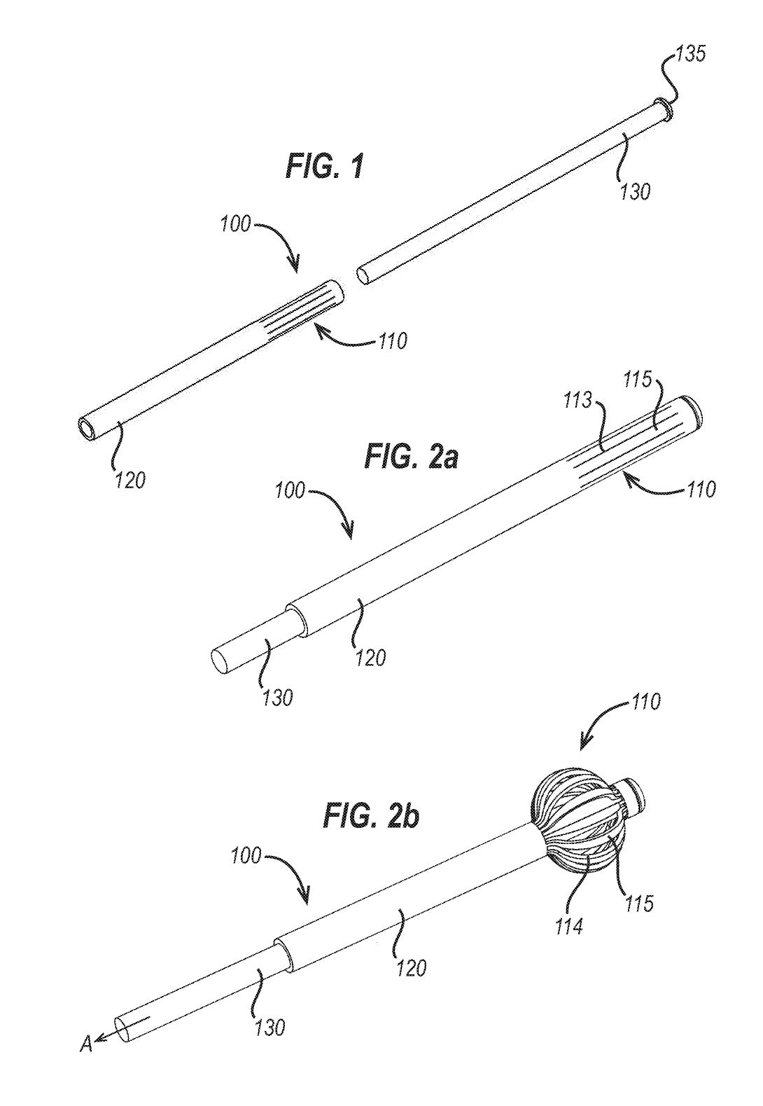

FIGS. 1, 2a and 2b show an exemplary embodiment of the clinical sampling device 100 of the present invention. FIG. 1 shows an exploded view of the parts of the clinical sampling device 100 which comprises a deformable sampling section 110, an incompressible sheath section 120 and a puller member 130. The puller member further comprises a stop 135 at its distal end, in the form of a flange of substantially the same diameter as the distal end of the deformable sampling section 110. In this example, the sheath 120 and deformable sampling section are formed as one integral element, in the form of a hollow cylindrical tube.

FIG. 2a shows the clinical sampling device 100 having been assembled by inserting the puller member 130 into the distal end of the sampling section 110, such that it extends through the sampling section 110 and sheath 120.

The sampling section 110 comprises a series of fins 115 which are joined at their distal end and their proximal end but are separable therebetween. In this example, these fins have been formed in the sampling section by a number of slits 113 which run, in a longitudinal direction, along the sampling section 110 and are arranged around the circumference of the sampling section 110.

The clinical sampling device 100 is movable between an undeployed configuration, shown in FIG. 2a, and a deployed configuration, shown in FIG. 2b. In the deployed configuration, the puller member has been moved in a proximal direction, relative to the sheath 120 and proximal end of the sampling section 110, as illustrated by arrow A. Equivalently, the sheath 120 and proximal end of the sampling section 110 can be moved in a distal direction relative to the puller member 130.

FIG. 2b shows the deployed configuration of the device, wherein the puller member 130 has been moved proximally so that stop 135 acts upon the distal end of the sampling section 110. As the sheath 120 is substantially incompressible, or at least has a longitudinal compressibility less than that of the deformable sampling section 110, the sampling section 110 has been compressed along its longitudinal axis, causing the fins 115 to deform and separate from each other between their distal and proximal ends. As such, the deformable sampling section 110 can be seen to have, around its mid-point, expanded radially, that is, it has expanded away from the longitudinal axis of the device. In other words, in the deployed configuration, the longitudinal length of the sampling section 110 has been decreased (relative to the undeployed configuration) whilst its width, perpendicular to the longitudinal axis, has increased (relative to the undeployed configuration).

In this, or any other example, the deformable sampling section 110 may be formed of a resilient material, such that in the deployed configuration, as the sampling section 110 is under compression it will exert a slight force upon the distal end of the puller member 130 and the distal end of the sheath 120, urging them apart. In this way, the device 100 is biased to its undeployed configuration, and when the puller member 130 is released, the device 100 will return to its undeployed configuration.

Of course, the deformable sampling section 110 may be configured such that it biases the device to the deployed configuration, such that it urges the distal end of the sampling section 110 towards the distal end of the sheath, or may be configured such that it does not bias the device 100 to either configuration. Alternatively, or additionally, separate biasing means may be provided to bias the device to its undeployed or deployed configuration.

As shown in FIG. 2b, as a result of the radial expansion of the fins 115, the edges 114 of the fins are exposed. Of course, the edges 114 of the fins will, in the undeployed configuration shown in FIG. 2a, abut the edge 114 of the neighbouring fin, and, as a result, will not be exposed.

Although the examples illustrated throughout the figures have a plurality of fins, it is also contemplated that the deformable sampling section may have only one fin. This may, for example, be arranged in a helical manner around the longitudinal axis.

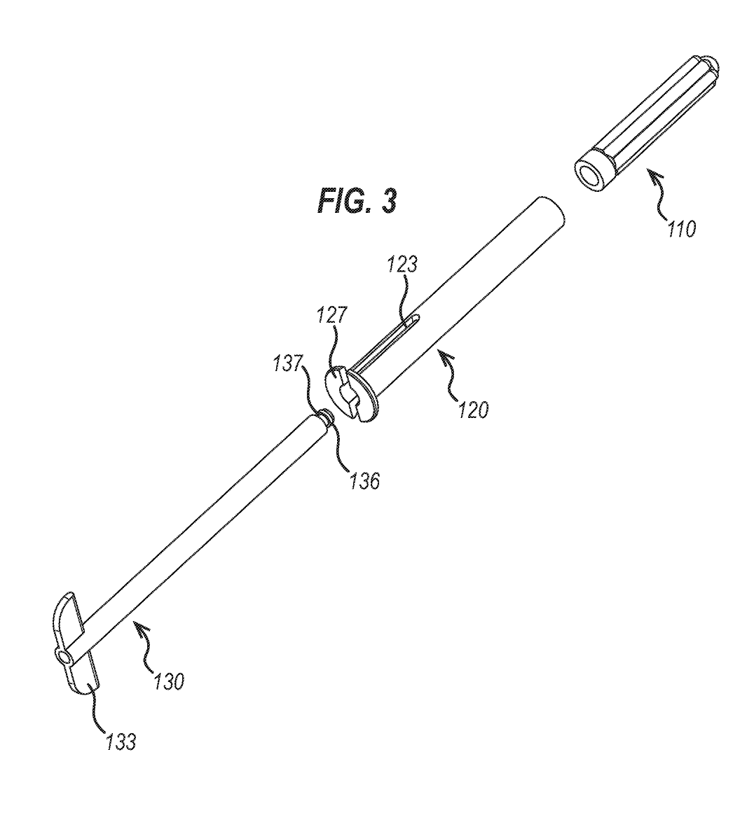

FIG. 3 shows a further exemplary embodiment of the tissue sampling device of the present invention. As can be seen, in this example, rather than being integrally formed together, the sheath 120 and deformable sampling section 110 are provided as separate pieces. Puller member 130 is provided with a lug 136 and channel 137 at its distal end for releasably attaching the puller member inside the distal end of the deformable sampling section 110. The device can thus be assembled by inserting the puller member into the proximal end of the sheath 120. The sampling section 110 is then placed over the distal end of the puller member 130.

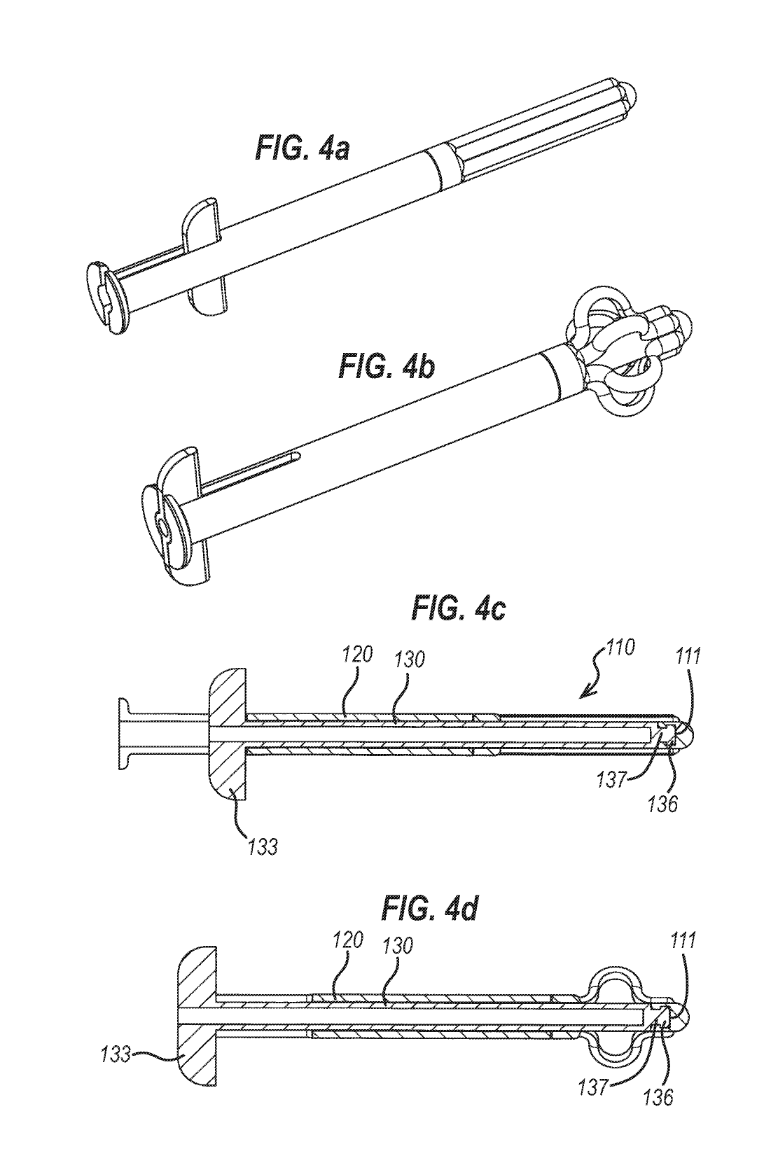

FIGS. 4a and 4b show the device of FIG. 3a, having been assembled, respectively showing its deployed and undeployed configuration. As most clearly shown in the corresponding cross-sectional views shown in FIGS. 4c and 4d, the distal end of the puller member 130 is received by a formation 111 in the distal end of the sampling section, whose profile corresponds to that of the lug 136 and channel 137, so as to releasably secure the distal end of the sampling section 110 to the distal end of the puller member 130. As a result, the sampling section is held on the puller member 130, in a position distal to the distal end of the sheath 120. The assembled tissue sampling device is shown in FIG. 4a.

Thus the deformable sampling section 110 is releasable attached to the rest of the device and is removable, by way of its releasable attachment to the puller member 130, and being separate from (i.e. not integral with) the sheath 130. Of course, the sampling section could alternatively be integral with the sheath 120 but be releasable from the sheath 120 by way of, for example, a frangible connection between the sheath 120 and the deformable sampling section 110.

As can be seen in FIGS. 2, 4a and 4b, the puller member 130 is provided with a pair of protrusions 133 which, as will be discussed in detail below, enable a practitioner to move the puller member and operate the device. It will of course be appreciated that a different number of protrusions may be provided. Each protrusion 133 is received by a corresponding slot 123 which extends longitudinally along the sheath 120, from its distal end, so that the protrusion 133 may extend from the body of the puller member 130 to outside the sheath 120. The protrusions 133 may be formed integrally with the body of the puller member 130, or may be formed as separate pieces and attached to the body of the puller member 133 by bonding through the use of adhesives or welding, for example, or otherwise connected to the body of the puller member 130.

Sheath 120 terminates in a flange 127, which extends radially from the body of the sheath 120.

As can be seen, in this example, the body of the sheath 120 is substantially formed of a hollow cylindrical tube, whilst the detachable sampling section 110 is, unlike the previous example, not formed of cylindrical tube with fins formed between slits.

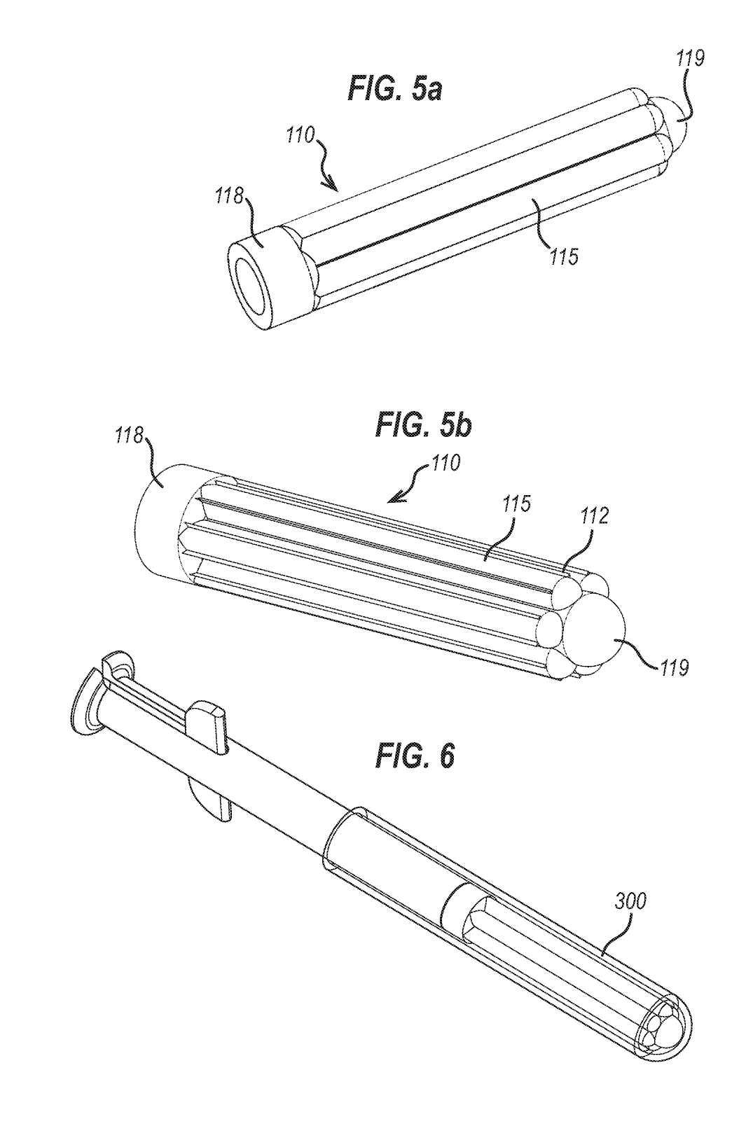

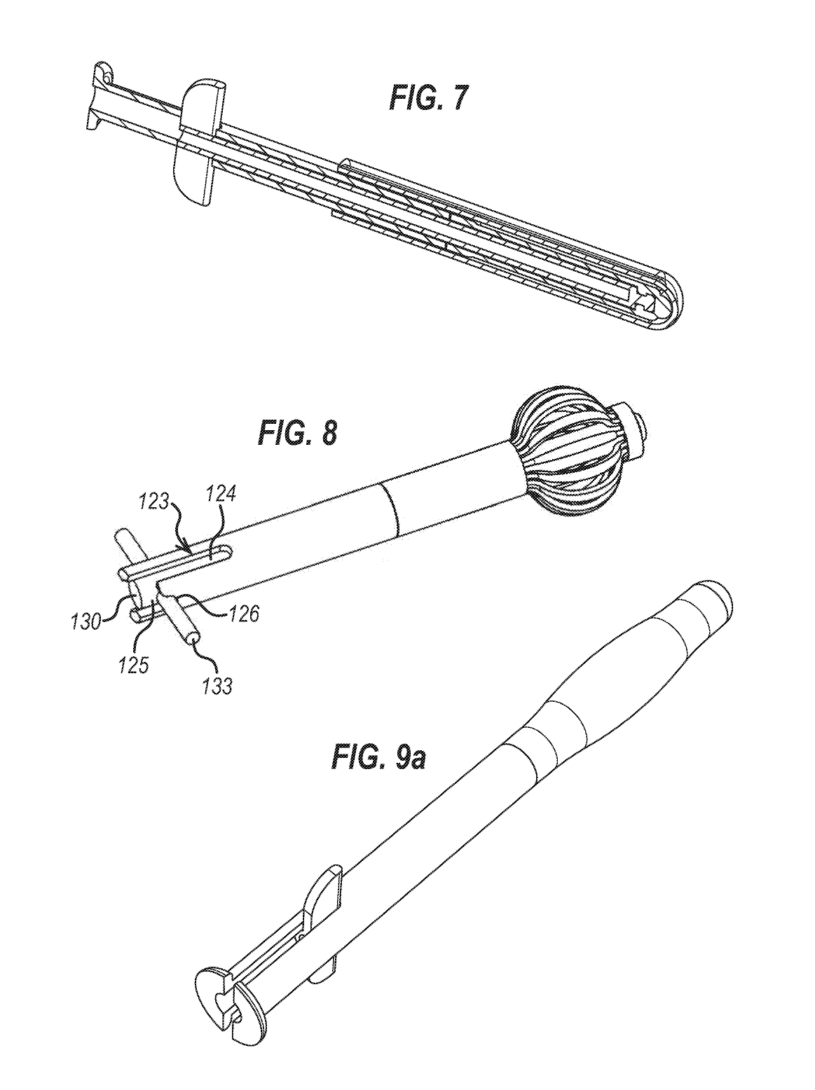

As shown in FIG. 5, the sampling section is formed with a number of independent fins 115 arranged in a cylindrical manner around the longitudinal axis of the sampling section 110. Each of the fins 115 has a semi-cylindrical cross-section. As in the previous example, the fins 115 are joined at their distal ends and proximal ends. The proximal end of the sampling section 110 comprises a hollow cylindrical section 118 with an outer diameter substantially the same as the outer diameter of the body of sheath 120. The proximal end of each of the fins 115 is connected to the proximal cylindrical section 118.

The distal end of the sampling section 120 terminates at a hemispheric section 119, and the proximal ends of each of the fins 115 is connected to the distal hemispheric section 119. As can also been seen, each of the fins 115 terminates, at its distal end, in a half-hemisphere.

The deformable sampling section 120 (including cylindrical portion 118, fins 115 and distal section 119) may be formed through, for example, injection moulding the sampling section 120 as a single integral piece. Alternatively, the sampling section 120 may be formed of a number of parts and then bonded together.

FIG. 5b shows another exemplary sampling section 120, which is similar that shown in FIG. 5a. However, in this example, each fin 115 has a number of ribs 112 which extend longitudinally along the fin. In this example, each fin 115 has two ribs 112, although it will be appreciated that a different number of ribs 112 may be provided on each fin 115. Furthermore, different fins 115 may have a different number of ribs 112, or some fins 115 may have no ribs 112.

The tissue sampling device 100 may be provided with a vessel 300 as shown in FIG. 6. The vessel 300 is dimensioned so as to receive the deformable sampling section 110.

A further exemplary embodiment of the tissue sampling device 100 is shown in FIG. 8. In particular, in this example the slot 123 is configured in a “bayonet fitting” type configuration. The slot 123 comprises a first, generally straight, portion 124 which extends longitudinally along the sheath 120 before turning to extend circumferentially around the sheath at circumferential portion 122. Similar to the example described above, the puller member 130 can be moved by way of protrusions 133 which are attached to the body of the puller member 130 and extend away from the longitudinal axis of the device 100 in a generally radial direction and extend through the slots 123. The puller member 130 can be moved first in a proximal direction along the longitudinal axis, each protrusion 133 is guided along the straight portion 124 of its corresponding slot 123. Once the puller member has been sufficiently withdrawn such that the protrusion is past the proximal end of the strait portion 124, the puller member can be rotated about its longitudinal axis, relative to the sheath, so as to move the protrusion along the circumferential portion 125. The circumferential portion terminates in a divot 126 that extends distally in the longitudinal direction. As the deformable sampling section 110 is resilient, it will bias the puller member 130 in the distal direction. As a result, on reaching the end of circumferential section 126, the protrusion 133 will move in a distal direction to be received by divot 126. In this way, the device 100 is “locked” in its deployed configuration. Of course, the device can be “unlocked” by first moving the protrusion 133 (and hence the puller member 130) proximally, such that the protrusion is past divot 126, and then rotating the puller member 130 to return the protrusion 133 to the straight portion 124. The puller member 130 can then move distally either under the action of the resilient deformable sampling section 110, to return the device to its undeployed configuration.

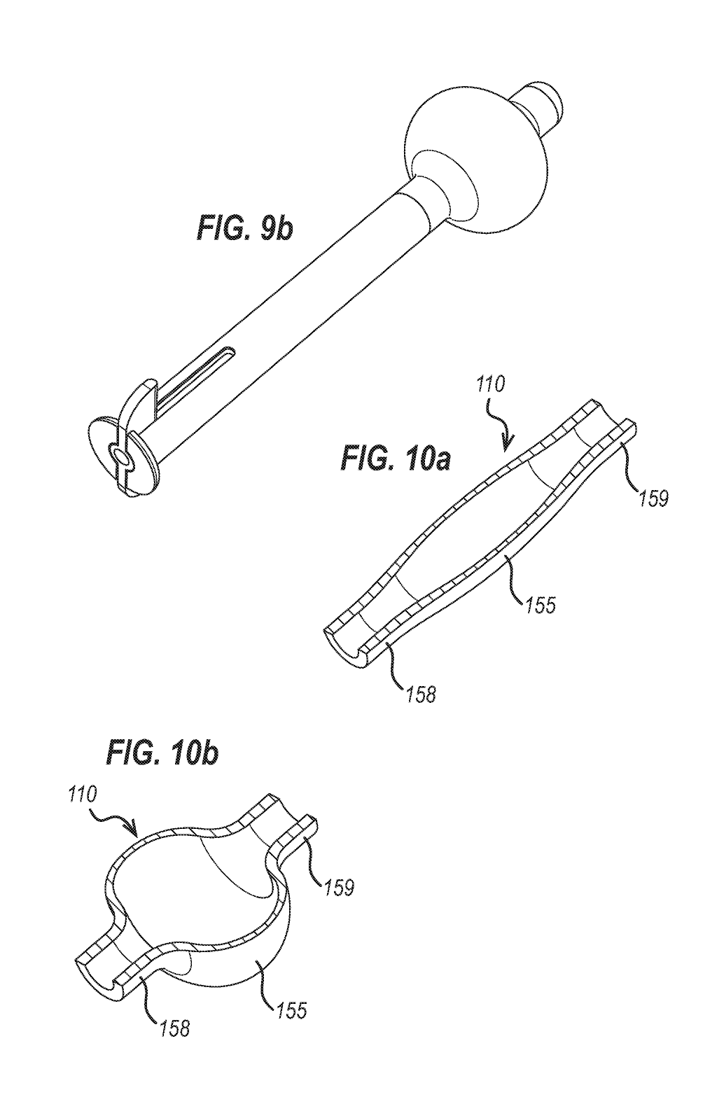

FIGS. 9a and 9b show an example of the tissue sampling device 100 which is similar in most respects to that shown in FIGS. 4a and 4b but in which the deformable sampling section 150 does not comprise a number of fins. In this example, deformable sampling section 150 instead comprises a continuous, roughly cylindrical hollow tube, formed of a deformable material wherein the walls of the tube have a variable deformability along the length of the deformable section. For example, FIGS. 10a and 10b show cross-sectional views of deformable sampling section 150 in the deployed and undeployed configuration of the device, respectively. As can be seen in FIG. 10a, the walls of the centre portion 155 of the sampling section 150 have a thickness which is less than that of the proximal portion 158 and distal portion 159.

Because the sheath 120 of the device is substantially incompressible, moving the puller member 130 in the proximal direction will result in the distal end of the puller member acting upon the distal end of the sampling section 150. The deformability of the walls of the sampling section 150 along the length of the sampling section will be dependent upon the thickness of the walls, being most deformable where the walls have the least thickness, and least deformable where the walls have the greatest thickness. As the deformability of the sampling section 150 is greatest at its centre portion 155, the centre portion 155 will expand radially outwards (that is, away from the longitudinal axis of the device) as the sampling section is compressed between the distal end of the puller member 130 (which acts upon the distal end of the sampling section 110) and the sheath 120.

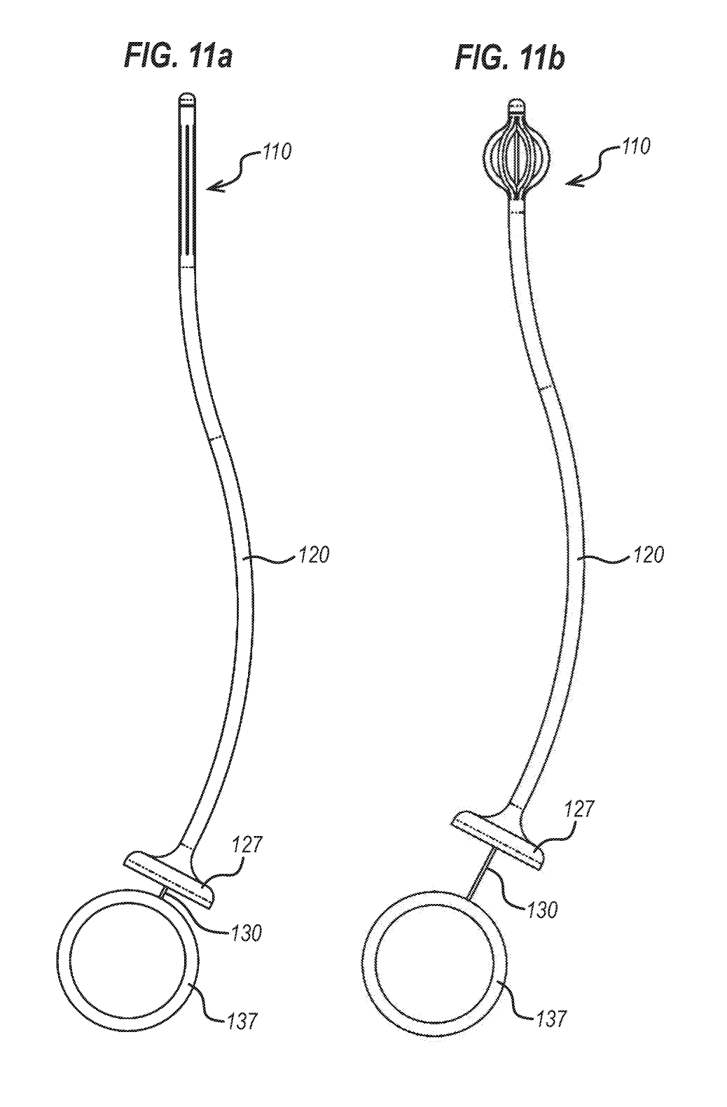

FIGS. 11a and 11b show another example of a clinical sampling device 100 which is configured as a flexible catheter. In this example, sheath 120 is an extended cylindrical tube which is flexible but which is substantially incompressible along its longitudinal axis. The puller member, in this case, is a flexible wire or similar, extends through the hollow sheath 120 and deformable sampling section 110, and is connected at its distal end to the distal end of the deformable sampling section.

As the sheath 120 is substantially incompressible, and the puller member 130 is substantially inextendible, when puller member 130 is moved in a proximal direction relative to the sheath 120 the device will move to its deployed configuration shown in FIG. 11b. In this configuration, the distal end of the puller member acts upon the distal end of the deformable sampling section 110, so that the sampling section 110 is compressed in a longitudinal direction. As a result, the deformable sampling section 110 is expanded in a radial direction.

This example also illustrates an alternative arrangement for the proximal end of the device. As in the example shown in FIGS. 4a and 4b, the sheath 120 has at its proximal end a flange 127. The puller member 130 has, at its proximal end, a ring 137.

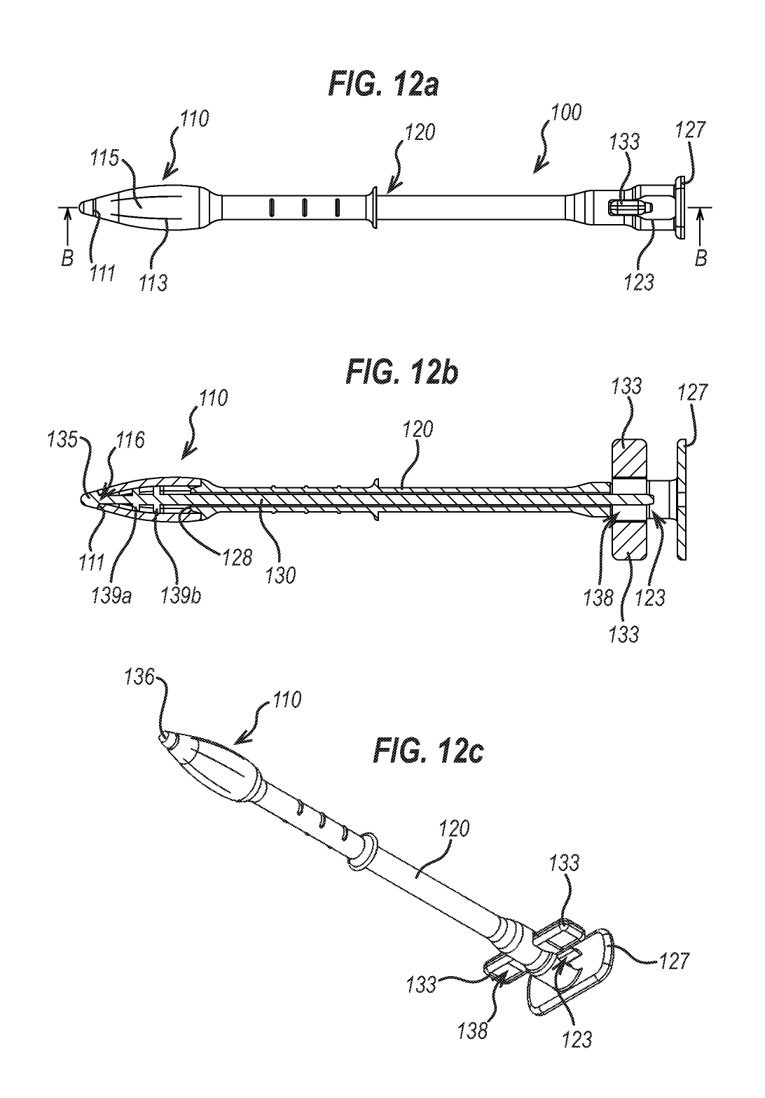

FIGS. 12a to 12c show an example of the tissue sampling device 100 which is similar in most respects to that shown in FIGS. 3 and 4a to 4d. In this embodiment, the sampling section 110 has a tapered configuration, narrowing toward its distal end. The sampling section 110 is divided into a plurality of fins 115 separated by slits 113.

In this example, puller member 130 comprises a stop 135 at its distal end which is received by formation 111 surrounding the open distal end 116 of the sampling section 110. Since the sampling section 111 is formed of a resilient but deformable material, the puller member 130 can be inserted into the proximal end of the sampling section 110, and stop 135 can be inserted, through the open distal end 116 of the sampling section, to be received by formation 111. A firm attachment is therefore provided between the distal end of the sampling section 110 and the distal end of the puller member 130. Proximal movement of the puller member 130 will thus cause the sampling section to be compressed longitudinally, and cause the sampling section to expand radially as the fins 115 deform and separate, as described above.

As the sampling section 110 is deformable, the attachment is releasable (as will be described in more detail below).

As shown in this example, protrusions 133 might be provided as part of a finger grip plate 138 which is attaches to the puller member 130 at its proximal end.

Additionally, puller member 130 is provided with flanges 139a, 139b positioned within the sampling section and configured to support the inner walls of the sampling section 110 to provide increased rigidity in its undeployed configuration and help maintain its shape. Additionally, the proximal-most flange 139b provides a stop surface which abuts a corresponding surface 128 at the distal end of the sheath 120 when the puller member 130 is moved proximally to prevent the sampling section 110 being over-compressed.

The use of the clinical sampling device 100 to obtain clinical samples from a sample site will now be described.

In this particular example, the use of the clinical sampling device 100 to obtain a clinical sample comprising cells from a sample site in the rectal cavity of a patient will be described. However, it will be understood that the clinical sampling device 100 might similarly be used to obtain other types of clinical samples from other sample sites in the human or animal body. Clinical samples may comprise tissues, cells or cellular material; micro-organisms such as bacteria, fungi and/or viruses; or biomarkers comprising nucleic acids and/or polypeptides, for example.

Before being used, the device must first be assembled. As shown in FIG. 3, and described above, the device may be in the form of sheath 120, puller member 130 and detachable sampling section 110. This is assembled by inserting the puller member 130 into the sheath 120 from the proximal end of the sheath 120. The sampling section 110 is then placed over the distal end of the puller member 130. The lug 136 and channel 137 disposed at the distal end of the puller member 130 will be received by a pocket with a corresponding profile within the distal end of the sampling section 110. As the sampling section is formed of a resilient but deformable material, this will result in a firm attachment between the distal end of the puller member 130 and the distal end of the sampling section 110.

In the example show in FIGS. 12a to 12c, the puller member 130 might first be attached to the sampling section 110 by inserting the puller member 130 through the proximal end of the sampling section 110 such that the stop 135 is forced through the open distal end 116 of the sampling section 110 and received by receiving formation 111. Finger grip plate 138 is inserted through slot 123 of the sheath 120. The sampling section 110 and puller member 130 can be assembled to the rest of the device by inserting puller member 130 into the distal end of the sheath 120 until a connecting element (not shown) at the proximal end of puller member 130 is received by, and attaches to, finger grip plate 138.

This assembly can take place before the device 100 is supplied to the practitioner who will use the device to collect a clinical sample. Alternatively, the device 100 may be supplied in a disassembled form, and assembled by the practitioner who will use the device to collect a clinical sample. Of course, the device 100 may also be supplied in a part-assembled form, for example, the puller member 130 may be inserted into the sheath 120 before the device is supplied to the practitioner, so that all that remains is to place sampling section 110 over the distal end of the puller member 130.

Once assembled, the device 100 is ready for use in obtaining a clinical sample from the rectal cavity of a patient. In its undeployed configuration, shown in FIG. 4a, the distal end of the device is inserted into a rectal cavity.

Insertion of the device 100 is aided by the fact the deformable sampling section 110 has a small width in the undeployed configuration, generally less than 1.5 cm. Further, as can be seen in FIG. 5a, the sampling section terminates, at its distal end, in hemispheric section 119, and each of the fins 115 terminates, at its distal end, in a half-hemisphere. These rounded leading surfaces of the device help to facilitate its insertion.

Once inserted, the puller member 130 can be moved in a proximal direction relative to the sheath 120 to compress the sampling section 110 in a longitudinal direction. In this example, the practitioner can place their thumb on surface 127 to hold the sheath 120 in place, whilst placing their fingers on the pair of protrusions 133 to pull on the protrusion 133, pulling the puller member 130 in a proximal direction.

Equivalently, the practitioner may push the sheath 120 in a distal direction by pushing on surface 127 whilst holding the puller member 130 in place by way of protrusions 133.

In either case, the puller member 130 will move proximally in a longitudinal direction relative to the sheath 120, and relative to the proximal end of sampling section 110. As such, the distal end of sampling section 110 will move towards the proximal end of the sampling section 110, causing the sampling section 110 to become compressed along its longitudinal axis as the device is moved into its deployed configuration.

As the deformable sampling section 110 is compressed longitudinally, it will correspondingly expand radially, that is to say, the sampling section will, about its midpoint along the longitudinal axis, expand away from the longitudinal axis. Thus, in its deployed configuration, the sampling section 110 has a greater width than in its undeployed configuration. In this example, and as shown in FIG. 4b, in the deployed configuration the fins 115 separate from each other as they expand radially.

By moving the device 100 into its deployed configuration such that the sampling section 110 expands radially, the deformable sampling section 110 will contact the walls of the rectal cavity so as to contact the sample site and collect a sample of cells. Once in its deployed configuration inside the cavity, the practitioner may rotate device 100 about its longitudinal axis to aid exfoliation of cells from the walls of the rectal cavity.

Once the wall of the rectal cavity has been exfoliated through contact with the deformable sampling section 100 with the device in its deployed configuration, the device 100 may be moved back into its undeployed configuration. As, in this example, the sampling section 110 is formed of a deformable but resilient material, releasing the grip on the protrusions 133 will cause the puller member 130 to move in a distal direction relative to the sheath under the biasing effect of the deformable sampling section 110, providing a force between the distal end of the puller member 130 and the distal end of the sheath 120. Once the device 100 has been returned to its undeployed configuration, the device may be withdrawn from the rectal cavity.

In the example shown in FIG. 5a the fins 115 have semi-cylindrical cross-section in the main, which, as discussed above, aids in the insertion of the device into the patient. The surface of the fins will exfoliate the sample site to obtain the sample of cells, but this exfoliation will be aided by edge 117. In examples such as those shown in FIGS. 2a and 2b, the fins 115 have a quadrilateral cross-section thus a similar function is provided by the edge 114 of each fin 115. In either case, in the undeployed configuration, the edge 117 or edge 114 abuts a corresponding edge of an adjacent fin. In this way, a clinical sample collected on the edge 117 or edge 114 will be captured between the fins, aiding the retention of the clinical sample on the sampling section 110 as the device 100 is withdrawn from the rectal cavity.

FIG. 5b shows a deformable sampling section which includes, on each of the fins 115, a pair of ribs 112, extending longitudinally along the fins 115. The ribs 112 may further aid any exfoliation of cells from the cavity walls when the device 100 is deployed within a rectal cavity.

FIGS. 9a and 9b show an example of the device in which the deformable sampling section 150 takes the form of a hollow, roughly cylindrical, tube with variable deformability along its longitudinal axis. As such, it provides a single continuous sampling surface. Of course, the surface of the sampling section may be textured to aide exfoliation. Additionally, or alternatively, the surface may be provided with cuts (not shown) which extend partially into the depth of the walls from the sampling edge, such that as the sampling section 150 expands radially, in the deployed configuration of the device, sampling edge surfaces are exposed to aid the exfoliation of cells, and also to retain said cells when the device is returned to its undeployed configuration, causing the cuts to close and the edge surfaces to no longer be exposed, aiding retention of the sample of cells on the sampling section as the device is withdrawn. The deformable sampling section 150 might additionally or alternatively be provided with ribs (not shown) to aid exfoliation.

Once the device 100 has been withdrawn, the deformable sampling section 110, on which the sample of cells has been collected, can be removed from the rest of the device 100.

In particular, it is desirable to be able to remove the sampling section 110 from the rest of the device without having to handle the sampling section 110. To this end, and as shown in FIG. 6, the device 100 may be provided with a vessel 300, dimensioned to receive the sampling section 110. That is to say, the vessel 300 is dimensioned to have an interior width greater than the width of the sampling section 110 when the device is in its undeployed configuration, but has an interior width less than that of the sampling section 110 when the device is in its deployed configuration. This facilitates convenient and sanitary removal of the sampling section 110 from the device 100, as will now be described.

To remove the sampling section 110 from the device, the sampling section 110 is inserted into the vessel 300. The puller member 130 is moved proximally (as described above) to cause the sampling section 110 to expand radially until it abuts the inner walls of the vessel 300. By applying continued force to urge the puller member 130 proximally with respect to the sheath 120, the deformable sampling section 110 will engage with the vessel 300, and be retained within the vessel. By pulling the puller member 130 proximally with sufficient force, the lug 136 and channel 137 will disengage from the receiving formation 111 in the distal end of the sampling section 110, whilst the sampling section 110 is retained within the vessel. The sampling section 110 will then no longer be attached at its distal end to the puller member 130 and the puller member 130 and sheath 120 can be removed, with the sampling section 110 remaining within the vessel 300.

Similarly, in the example shown in FIGS. 12a to 12c, the sampling section 110 can be inserted into a suitably shaped vessel and the puller member 130 moved proximally to cause the sampling section 110 to expand radially until it abuts the inner walls of the vessel. By pulling the puller member 130 and sheath 120 proximally with sufficient force, the stop 135 will be withdrawn through the open distal end 116 of the sampling section 110 and disengage with receiving formation 111. The sampling section 110 will then no longer be attached at its distal end to the puller member 130 and the puller member 130 and sheath 120 can be removed, with the sampling section 110 remaining within the vessel.

In this way, it is possible to detach sampling section 110 from the device 100, without having to handle the sampling section 110. Not only is this more sanitary, but mitigates the possibility of contaminating the clinical sample which has been collected on the sampling section 110. Furthermore, the sampling section 110 can be removed from the device 100 using only one hand which is convenient for the user, and with the hand being distant from the sampling section so as to reduce the risk of contamination even further.

Once the sampling section 110 has been detached from the device 100, and the device 100 removed, the vessel 300 can be sealed by placing a cap over the end of the vessel 300. Before sealing the vessel 300, a suitable transport buffer may be introduced to the vessel 300, to preserve the sample of cells whilst, for example, the sample awaits analysis. Alternatively, a vessel 300 may be provided which already contains a suitable transport buffer.

The transport buffer preferably is provided so as to maintain the collected material in an intact state whilst the sample awaits analysis. The analysis may be then be performed at a different location to where the sample was collected. Preferably, the buffer should be formulated to inhibit bacterial growth and ensure that the sample is maintained in an appropriate state to prevent any degradation of the target analyte between collection and analysis.

As in this example, where the device is used to collect a sample comprising cells, the transport buffer might be formulated to preserve the cells, to prevent cell lysis, to inhibit bacterial growth, to prevent protein degradation, prevent nucleic acid degradation, or any combination of the above. It may contain, for example, a protease inhibitor, a nuclease inhibitor, salts, buffers, or any combination of the above.

The transport buffer might be provided in the form of a solution. Alternatively the buffer might be provided partly in the form of a solution, and partly in the form of a tablet which might be dissolved in the solution to make up a transport buffer solution before use. The solution might be provided in a collection tube so that a user can add the tablet part of the solution to the collection tube to dissolve said tablet and form the transport buffer. The sampling section 110 can then be introduced into the vessel 300. The sampling section 110 can then be removed from the device 100, and the vessel sealed. In this way, the collected sample can be maintained on the sampling section 110, within the transport buffer, whilst the sample awaits analysis.

Because the sampling section 110 is provided as a detachable, separate component, it is possible to supply a kit which comprises the puller member 130, sheath 120 and a number of sampling sections 110, each with corresponding vessel 300. A practitioner may then take a number of clinical samples using the same device, but replacing the detachable sampling section 110 to take each sample. Also, the kit may comprise a number of sampling sections 110 which have different configurations, for example, the kit may comprise sampling sections which have different degrees of deformability, have different widths in their deployed configuration, have different numbers of fins, or may comprise a number of sampling sections which have a continuous sampling surface (as per FIGS. 10a and 10b) and a number which comprise fins. The practitioner would then be able to select the sampling section which is most appropriate in view of, for example, the type of sample to be obtained from the patient, the location of the sample site within the patient, or the condition of the patient.

Moreover, a kit may be provided which comprises a number of sampling sections 110 and corresponding vessels 300, to be used with sheath 120 and puller member 130. In this way, if a practitioner is already in possession of an appropriate device (i.e. puller member 130 and sheath 120), replacement sampling sections may be provided.

The kit may optionally contain the transport buffer.

Use of the tissue sampling device has been described above in relation to obtaining a sample of cells from a rectal cavity. It will be apparent to the skilled person that a clinical sampling device may be configured to collect clinical samples from other locations within a patient, for example any body cavity including but not limited to the colon, the uterus, the cervix, the mouth, the oesophagus, the trachea, the intestine, the nasal passages, the urethra, or the bladder; any other part of the body; or any duct or vessel. For example, FIGS. 11a and 11b show a tissue sampling device which has been configured as a flexible catheter which can be introduced, through the rectum of a patient, to retrieve clinical samples from the colon. In this example, the sheath 120 is elongate and flexible, but substantially incompressible along its longitudinal axis. The puller member 130, formed of wire or similar, is flexible but inextendible in its longitudinal direction. As the tissue sampling device is flexible, it can be advanced through the patient's colon until it reaches the position from which a tissue sample is to be collection. The puller member is then moved proximally by pulling ring 137 proximally, relative to flange 127. The distal end of puller member 130, which is operable attached to the distal end of the sampling section 110, will act upon the sampling section 110 to compress it longitudinally against the distal end of the substantially incompressible sheath 120. As described above, the sampling section 110 will expand radially as the tissue sampling device is moved into its deployed configuration. A tissue sample can be obtained, as described above, before the device 100 is returned to its undeployed configuration and withdrawn from the patient.

It will of course be understood that the embodiments of the present invention have been described above purely by way of example and modifications of detail can be made within the scope of the invention.

1. A device for collecting a clinical sample by contacting a sample site, movable between an undeployed configuration and a deployed configuration, the device comprising:
a deformable sampling section extending along a longitudinal axis;
wherein in the undeployed configuration the deformable sampling section has a first length in the longitudinal direction and a first width in a radial direction, perpendicular to the longitudinal axis, and
in the deployed configuration, the deformable sampling section has a second length, shorter than the first length and at least part of the deformable sampling section has second width, greater than the first width, so as to contact the sample site.

2. A device for collecting a clinical sample by contacting a sample site, movable between an undeployed configuration and a deployed configuration, the device comprising:
a deformable sampling section extending along a longitudinal axis and comprising at least one sampling edge;
wherein in the undeployed configuration the deformable sampling section has a first width in a radial direction, perpendicular to the longitudinal axis, and the at least one sampling edge is not exposed and cannot contact the sample site; and
in the deployed configuration, at least part of the deformable sampling section has a second width, greater than the first width, such that the at least one sampling edge is exposed and can contact the sample site.

3. The device of any preceding claim, wherein the sampling section comprises at least one fin.

4. The device of any preceding claim, wherein the sampling section comprises a plurality of fins, which are attached at their distal ends, are attached at their proximal ends, and which are separable therebetween.

5. The device of claim 3 or claim 4, when dependent upon claim 1, wherein the at least one fin or at least one of the plurality of fins comprises a sampling edge which, when the device is in its deployed configuration, is exposed and, when the device is in its undeployed configuration, is not exposed.

6. The device of claim 3 or claim 4, when dependent upon claim 2, wherein the at least one fin or at least one of the plurality of fins comprises the sampling edge.

7. The device of claim 6, wherein the sampling edge is disposed on an edge of the fin, extending longitudinally.

8. The device of claim 7, wherein, when the device is in its undeployed configuration, the sampling edge abuts a second sampling edge.

9. The device of claim 8, wherein the second sampling edge is disposed on a second fin of the plurality of fins.

10. The device of any one of claims 3 to 9, wherein the at least one fin or at least one of the plurality of fins comprises at least one rib.

11. The device of claim 10, wherein the at least one rib extends longitudinally along the fin.

12. The device of any preceding claim, wherein in the undeployed configuration, the sampling section has a generally cylindrical configuration.

13. The device of any preceding claim, further comprising a puller member which at least partially extends through the sampling section along the longitudinal axis.

14. The device of claim 13, wherein the puller member is longitudinally inextendible.

15. The device of claim 13 or claim 14, wherein the puller member cooperates with the distal end of the sampling section, so as to act upon the sampling section and longitudinally compress the sampling section when the puller member is moved in a proximal direction relative to the proximal end of the sampling section.

16. The device of any one of claims 13 to 15, wherein the sampling section is detachable from the puller member.

17. The device of any preceding claim, further comprising a sheath, proximal to the sampling section.

18. The device of claim 17, when dependent upon claim 13, wherein the puller member also extends at least partially through the sheath.

19. The device of claim 17 or claim 18, wherein the sheath has a longitudinal compressibility less than that of the sampling section.

20. The device of claim 19, wherein the sheath is longitudinally incompressible.

21. The device of any one of claims 17 to 20, wherein the sheath and at least part of the sampling section have different thicknesses.

22. The device of any one of claims 17 to 21, wherein the sheath is integral with the sampling section.

23. The device of any of claims 17 to 21, wherein the sampling section is separate from the sheath.

24. The device of any of claims 17 to 23, wherein the sampling section is detachable from the sheath.

25. The device of any of claims 17 to 24, wherein the proximal end of the sheath comprises a flange.

26. The device of any of claims 17 to 25, when dependent upon claim 13, wherein the sheath comprises at least one slot with a first portion which extends longitudinally along the sheath and the device further comprises at least at least one protrusion which extends radially outwards from the puller member, through the slot, and is connected to the puller member.

27. The device of claim 26, wherein the slot further comprises a second portion extending circumferentially from the first portion.

28. The device of claim 27, wherein the slot and protrusion are arranged to be able to retain the device in its deployed configuration.

29. The device of claim 27 or claim 28, wherein the slot and protrusion are arranged to be able to retain the device in its undeployed configuration.

30. The device of any one of claims 17 to 29, wherein the sheath is flexible.

31. The device of any one of claims 13 to 16, or any one of claims 17 to 19 when dependent upon claim 13, wherein the puller member is flexible.

32. The device of any preceding claim where the deformable sampling section is configured to bias the device to its undeployed configuration.

33. The device of any preceding claim where the deformable sampling section is configured to bias the device to its deployed configuration.

34. The device of any preceding claim, wherein the device is configured as a flexible catheter.

35. The device of any preceding claim, wherein the device comprises a plurality of sampling sections.

36. The device of any preceding claim, wherein the sampling section comprises silicone, ethylene propylene diene terpolymer, thermoplastic elastomer or natural latex rubber.

37. The device of claim 23, or any of claims 24 to 36 when dependent upon claim 23, wherein the sheath comprises polypropylene, polystyrene or polyethylene.

38. The device of any preceding claim, wherein the puller member comprises polyethylene or polypropylene.

39. A deformable sampling section for use in a clinical sample collecting device, wherein, in use, the deformable sampling section has an undeployed configuration and a deployed configuration;
wherein in the undeployed configuration the deformable sampling section has a first length in the longitudinal direction and a first width in a radial direction, perpendicular to the longitudinal axis, and
in the deployed configuration, the deformable sampling section has a second length, shorter than the first length and at least part of the deformable sampling section has second width, greater than the first width, so as to contact a sample site.

40. A deformable sampling section for use in a clinical sample collecting device, wherein, in use, the deformable sampling section has an undeployed configuration and a deployed configuration;
wherein the deformable sampling section extends along a longitudinal axis and comprises at least one sampling edge; and
wherein in the undeployed configuration the deformable sampling section has a first width in a radial direction, perpendicular to the longitudinal axis, and the at least one sampling edge is not exposed and cannot contact tissue; and
in the deployed configuration, at least part of the deformable sampling section has a second width, greater than the first width, such that the at least one sampling edge is exposed and can contact a sample site.

41. A clinical sampling device adapted to connect to the deformable sampling section of claim 39 or claim 40, comprising:
a sheath; and
a puller member which at least partially extends through the sheath and is adapted to extend at least partially through the deformable sampling section, and the sheath and puller member are further adapted to act upon the deformable sampling section and longitudinally compress the sampling section when the puller member is moved in a proximal direction.

42. A kit comprising:
the deformable sampling section of claim 39 or claim 40, and
a collection vessel, dimensioned to receive the deformable sampling section.

43. The kit of claim 42, further comprising the device of claim 41.

44. The kit of claim 42 or claim 43, wherein the kit further comprises a transport buffer for cells.

45. The kit of claim 44, wherein the collection vessel of the kit contains the transport buffer.

46. The kit of any one of claims 42 to 45, wherein the collection vessel is dimensioned such that at least part of the vessel is wider than the sampling section when the device is in its undeployed configuration and narrower than the sampling section when the device is it its deployed configuration.

47. The kit of claim 46, wherein the deformable sampling section has a vessel engaging configuration wherein at least part of the deformable section expands radially outwards to engage the collection vessel.

48. A method of preparing a tissue sample for analysis, wherein the tissue sample is disposed on the sampling section of the device of any one of claims 16 or claim 24 or any of claims 25 to 38 when dependent upon either claim 16 or claim 24, the method comprising:
inserting the device into a collection vessel,
detaching the sampling section from the puller member and/or sheath.

49. The method of claim 48, wherein the step of detaching the sampling section from the puller member and/or sheath comprises expanding the at least part of the deformable sampling section to engage with the collection vessel.

50. The method of claim 48 or claim 49, further comprising, before the step of inserting the device into the collection vessel, the step of introducing a collection buffer into the collection vessel.

51. The method of claim 48 or claim 49, further comprising, after the step of detaching the sampling section from the puller member and/or sheath, the step of introducing a collection buffer into the collection vessel.
